(19) United States
(12) Patent Application Publication
(10) Pub. No.: US 2012/0197268 A1
(43) Pub. Date: Aug. 2, 2012

(54) SELF EYEBROW WAXING AND SHAPING SYSTEM

(76) Inventor: Janet Tran, Pearland, TX (US)

(21) Appl. No.: 13/334,262

(22) Filed: Dec. 22, 2011

Related U.S. Application Data
(60) Provisional application No. 61/437,867, filed on Jan. 31, 2011.

(51) Int. Cl. A61B 17/50

(52) U.S. Cl. 606/134

(57) ABSTRACT

Self eyebrow waxing and shaping system provides consumers with a method for independently creating and maintaining symmetrical eyebrow shapes. Self eyebrow waxing and shaping system comprises eyebrow shaping stencil template strip(s). Eyebrow shaping stencil template strip(s) may include a plurality of left eyebrow shaping stencil template strip(s); a plurality of right eyebrow shaping stencil template strip(s); an adhesive wax backing; a protective peel off sheet; and a pre-shaped eyebrow cutout. Each of left eyebrow shaping stencil template strip(s) and right eyebrow shaping stencil template strip(s) comprise the adhesive wax backing, the protective peel off sheet, and the pre-shaped eyebrow cutout. A left eyebrow shaping stencil template strip(s) is preferably removeably-placed over left eyebrow of user to alter a shape of left eyebrow while right eyebrow shaping stencil template strip(s) is preferably removeably-placed over right eyebrow of user to alter a shape of a right eyebrow.

### CROSS-REFERENCE TO RELATED APPLICATION

The present application is related to and claims priority from prior provisional application Ser. No. 61/437,867, filed Jan. 31, 2011 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of eyebrow shaping devices and more particularly pertains to a self eyebrow waxing and shaping system to create neatly shaped eyebrows.

2. Description of the Related Art

The eyebrow is an area of thick, delicate hairs located above the eye that follows the shape of the lower margin of the brow ridges. Their main function is to prevent sweat, water, and other debris from falling down into the eye socket, but they are also important to human communication and facial expression. Eyebrows are also a major facial feature. Cosmetic methods have been developed to alter the look of one's eyebrows, whether the goal is to add or remove hair, change the color, or change the position of the eyebrow.

Several options for removing hair to achieve a thinner or smaller eyebrow, or to correct a unibrow may include tweezing, waxing, threading and use of electronic tweezers. The most common method is to use tweezers to thin out and shape the eyebrow. Waxing is becoming more popular. Lastly, there is threading eyebrows, where a cotton thread is rolled over hair to pull it out. Small scissors are sometimes used to trim the eyebrows, either with another method of hair removal or alone. All of these methods can be painful for some seconds or minutes due to the sensitivity of the area around the eye but, often, this pain decreases over time as the individual becomes desensitized. In time, hair that has been plucked may stop growing back. Some people wax or shave off their eyebrows and may have a permanent tattoo applied in their place; however this may be a drastic move that may not be easily changed in subsequent years.

Unfortunately, many individuals may struggle to achieve a symmetrical, evenly-shaped eyebrow by plucking their own eyebrows. These individuals often utilize beauty salon or spa professionals to wax their eyebrows for them. However, this can be expensive, and therefore may not be a practical expense for most consumers. Also, many people like to pluck their own eyebrows between waxing appointments, to ensure optimal appearance of their eyebrow shapes. Even performing basic maintenance can be difficult and painful for some individuals. An effective solution is necessary whereby an individual can symmetrically manicure eyebrows.

Various attempts have been made to solve the above-mentioned problems such as those found in U.S. Pat. Nos. 7,322,991; 2004/0226572; 2003/0234028; 6,336,462; 2,917,058; and D601,749. This prior art is representative of kits for use with eyebrows. None of the above inventions and patents, taken either singly or in combination, is seen to describe the invention as claimed.

Ideally, a self eyebrow waxing and shaping system should be safe, user-friendly and, yet would operate reliably and be manufactured at a modest expense. Thus, a need exists for reliable a self eyebrow waxing and shaping system to avoid the above-mentioned problems.

### BRIEF SUMMARY OF THE INVENTION

In view of the foregoing disadvantages inherent in the known eyebrow shaping devices art, the present invention provides a novel self eyebrow waxing and shaping system configured specifically to create neatly shaped eyebrows. The general purpose of the present invention, which will be described subsequently in greater detail, is to provide a user with a method for independently creating and maintaining symmetrical eyebrow shapes.

A self eyebrow waxing and shaping system is disclosed herein in a preferred embodiment preferably comprising: rectangular eyebrow shaping stencil template strip(s) having a plurality of left eyebrow shaping stencil template strip(s); a plurality of right eyebrow shaping stencil template strip(s); an adhesive wax backing; a protective peel off sheet; and a pre-shaped eyebrow cutout. The eyebrow shaping stencil template strip(s) comprise a sheet of waxing material with adhesive backing.

Each of the left eyebrow shaping stencil template strip(s) and the right eyebrow shaping stencil template strip(s) comprise the adhesive wax backing, the protective peel off sheet, and the pre-shaped eyebrow cutout. The left eyebrow shaping stencil template strip(s) is removeably-placed over a left eyebrow of the user to alter a shape of the left eyebrow. The right eyebrow shaping stencil template strip(s) is removeably-placed over a right eyebrow of the user to alter a shape of the right eyebrow.

Further, the eyebrow shaping stencil template strip may comprise an elongated corner to permit ease of removing the protective peel off sheet thereby exposing the adhesive wax backing. The eyebrow shaping stencil template strips are preferably (substantially) transparent beige thereby permitting the user to visualize how much of the eyebrow will be removed upon completion of the self eyebrow waxing.

The pre-shaped eyebrow cutout may be located in a central portion on the eyebrow shaping stencil template strips. Each of the pre-shaped eyebrow cutouts may comprise a different shape and design on each eyebrow shaping stencil template strip. The pre-shaped eyebrow cutout comprises ten different eyebrow shape variations on eyebrow shaping stencil template strip. The pre-shaped eyebrow cutout is arched and contoured to a natural shape of the eyebrow with an outer end of the eyebrow being tapered.

The adhesive wax backing is preferably located on a bottom side of the eyebrow shaping stencil template strip. The adhesive wax backing on the eyebrow shaping stencil template strip removes unwanted hair and creates a desired shape of the eyebrow as per shape of the pre-shaped eyebrow cutout. The protective peel off sheet protects the adhesive wax backing disposed upon the bottom side of the eyebrow shaping stencil template strip and is peeled off prior to a user using the eyebrow shaping stencil templates.

A kit is embodied herein for the self eyebrow waxing and shaping system comprising: a plurality of left eyebrow shaping stencil template strips in various pre-shaped eyebrow cutout designs; a plurality of right eyebrow shaping stencil template strips in various pre-shaped eyebrow cutout designs; and a set of user instructions.

A method of using a self eyebrow waxing and shaping system is disclosed herein preferably comprising the steps of: choosing a left eyebrow shaping stencil template strip with a desired left pre-shaped eyebrow cutout design; grasping an elongated corner of the eyebrow shaping stencil template strip; peeling away a protective peel off sheet; placing the eyebrow shaping stencil template strip over a left eyebrow; pressing the eyebrow shaping stencil template strip onto skin around the left eyebrow of a user where unwanted eyebrow hair is located; allowing the adhesive wax backing to grab the unwanted eyebrow hair; and quickly peeling eyebrow shaping stencil template strip away from eyebrow to reveal a newly shaped eyebrow (on the left side.)

The method of using a self eyebrow waxing and shaping system further comprising the steps of: choosing a right eyebrow shaping stencil template strip with a desired right pre-shaped eyebrow cutout design; grasping an elongated corner of the eyebrow shaping stencil template strip; peeling away a protective peel off sheet; placing the eyebrow shaping stencil template strip over a right eyebrow; pressing the eyebrow shaping stencil template strip onto skin around the right eyebrow of a user where unwanted eyebrow hair is located; allowing adhesive wax backing to grab the unwanted eyebrow hair; and quickly peeling the eyebrow shaping stencil template strip away from the eyebrow to reveal a newly shaped eyebrow (on the right side.)

The present invention holds significant improvements and serves as a self eyebrow waxing and shaping system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, self eyebrow waxing and shaping system constructed and operative according to the teachings of the present invention.

FIG. 1 shows a perspective view illustrating an eyebrow shaping stencil template strip being applied to a face of a user of a self eyebrow waxing and shaping system according to an embodiment of the present invention.

FIG. 2 is a perspective view illustrating a direction (indication) in which the eyebrow shaping stencil template strip is to be quickly peeled away from a face of a user of the self eyebrow waxing and shaping system according to an embodiment of the present invention.

FIG. 3 is a perspective view illustrating a protective peel off sheet being peeled away from an adhesive wax backing on the eyebrow shaping stencil template strip of the self eyebrow waxing and shaping system according to an embodiment of the present invention.

FIG. 4 is a perspective view illustrating a kit of the self eyebrow waxing and shaping system according to an embodiment of the present invention.

FIG. 5 is a flowchart illustrating a method of use according to an embodiment of the present invention of FIGS. 1-4.

The various embodiments of the present invention will hereinafter be described in conjunction with the appended drawings, wherein like designations denote like elements.

### DETAILED DESCRIPTION

As discussed above, embodiments of the present invention relate to eyebrow shaping devices art, the present invention provides a novel self eyebrow waxing and shaping system configured specifically to create neatly shaped eyebrows. The general purpose of the present invention, which will be described subsequently in greater detail, is to provide a user with a method for independently creating and maintaining symmetrical eyebrow shapes

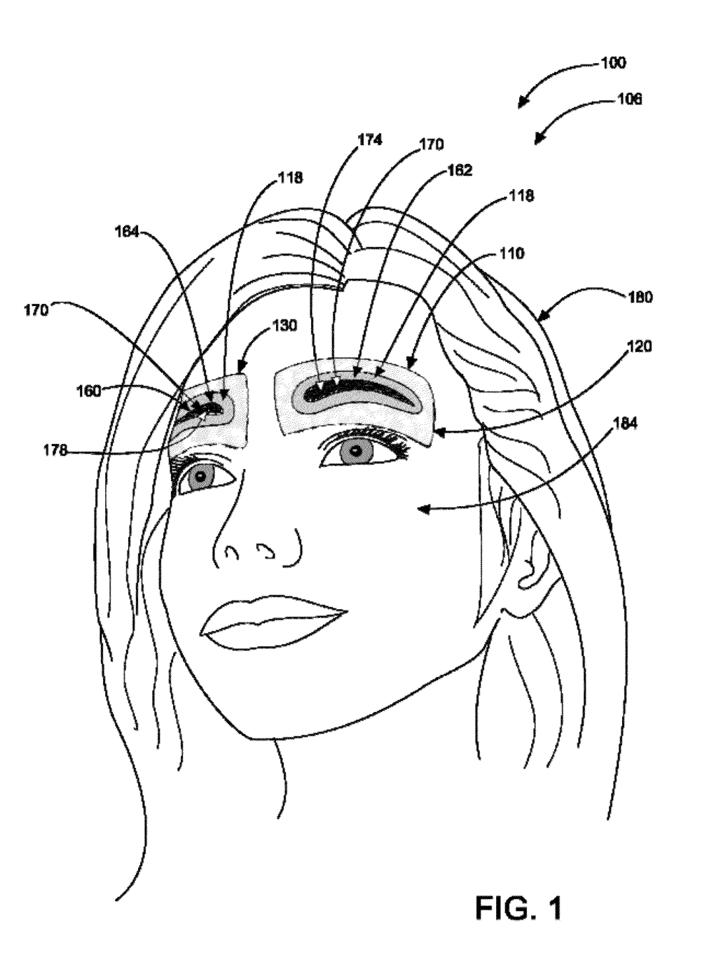

Referring to the drawings by numerals of reference there is shown in FIGS. 1 & 2 illustrating eyebrow shaping stencil template strip 110 being applied to face 184 of user 180 of self eyebrow waxing and shaping system 100 according to an embodiment of the present invention.

Self eyebrow waxing and shaping system 100 in a preferred embodiment preferably comprises: eyebrow shaping stencil template strip(s) 110. Eyebrow shaping stencil template strip(s) 110 preferably includes a plurality of left eyebrow shaping stencil template strip(s) 120; a plurality of right eyebrow shaping stencil template strip(s) 130; adhesive wax backing 140; protective peel off sheet 150; and pre-shaped eyebrow cutout(s) 160. Eyebrow shaping stencil template strip(s) 110 comprise protective peel off sheet 150 preferably with adhesive backing 110 for ease of use and manufacture. Self eyebrow waxing and shaping system 100 is usable to independently create and maintain symmetrical shapes of eyebrows 170.

Eyebrow shaping stencil template strip(s) 110 are preferably transparent beige thereby permitting user to visualize how much of eyebrow 170 will be removed upon completion of self eyebrow waxing and shaping system 100, however other colors may be manufactured such as brown or black or transparent. Additionally, eyebrow shaping stencil template strip(s) 110 are preferably rectangular in shape. Upon reading this specification, it should be appreciated that, under appropriate circumstances, considering such issues as user preferences, design preference, structural requirements, marketing preferences, cost, available materials, technological advances, etc., other shape arrangements such as, for example, oval, circular and the like may be sufficient.

Each of left eyebrow shaping stencil template strip(s) 120 and right eyebrow shaping stencil template strip(s) 130 preferably comprise adhesive wax backing 140, protective peel off sheet 150, and pre-shaped eyebrow cutout 160. Left eyebrow shaping stencil template strip(s) 120 is preferably removeably-placed over left eyebrow 174 of user 180 to alter a shape of left eyebrow 174. Right eyebrow shaping stencil template strip(s) 130 is preferably removeably-placed over right eyebrow 178 of user 180 to alter a shape of right eyebrow 178. It should be noted that eyebrow shaping stencil template strip(s) 130 may be manufactured in different sizes having both adhesive wax backing 140 and protective peel off sheet 150 to be used for waxing on other body portions of user 180 in addition to eyebrows 170 such as a chest region, arms, under arms, face 184 or around bikini lines.

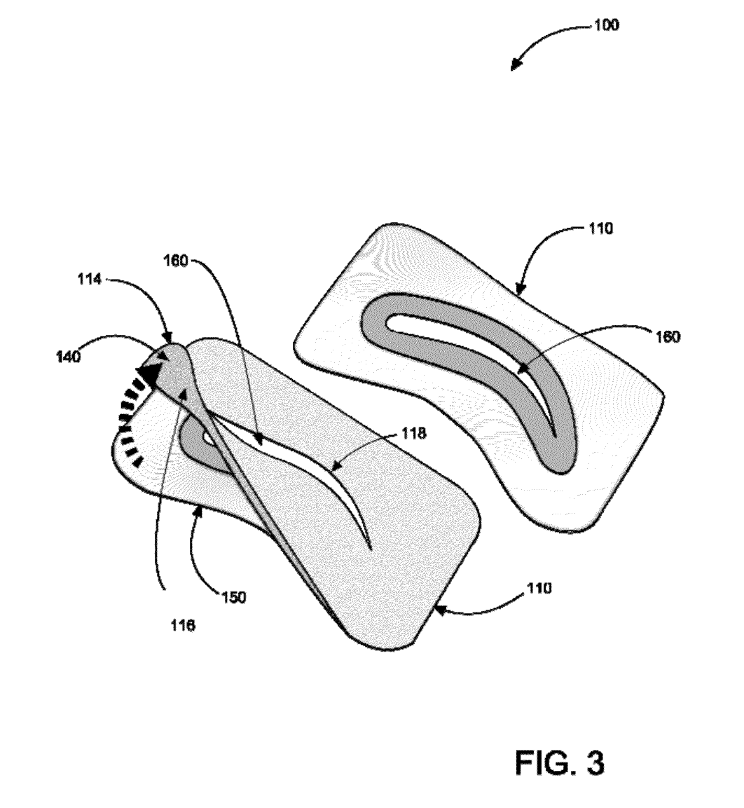

Referring now to FIG. 3 illustrating a perspective view illustrating protective peel off sheet 150 being peeled away from an adhesive wax backing 140 on eyebrow shaping stencil template strip(s) 110 of the self eyebrow waxing and shaping system 100 according to an embodiment of the present invention.

Eyebrow shaping stencil template strip(s) 110 preferably comprises elongated corner 114 to permit ease of removing protective peel off sheet 150 thereby exposing adhesive wax backing 140. Protective peel off sheet 150 is preferably manufactured from wax-paper to prevent protective peel off sheet 150 from sticking to adhesive wax backing 140. Upon reading this specification, it should be appreciated that, under appropriate circumstances, considering such issues as user preferences, design preference, structural requirements, marketing preferences, cost, available materials, technological advances, etc., other material arrangements such as, for example, other non-stick materials and papers may be sufficient.

Adhesive wax backing 140 is preferably located on bottom side 116 of eyebrow shaping stencil template strip(s) 110. Adhesive wax backing 140 on eyebrow shaping stencil template strip(s) 110 preferably removes unwanted hair and creates a desired shape of eyebrow 170 as per shape of pre-shaped eyebrow cutout 160. Protective peel off sheet 150 preferably protects adhesive wax backing disposed upon bottom side 118 of the eyebrow shaping stencil template strip(s) 110 and is peeled off prior to user 180 using of eyebrow shaping stencil template strip(s) 110.

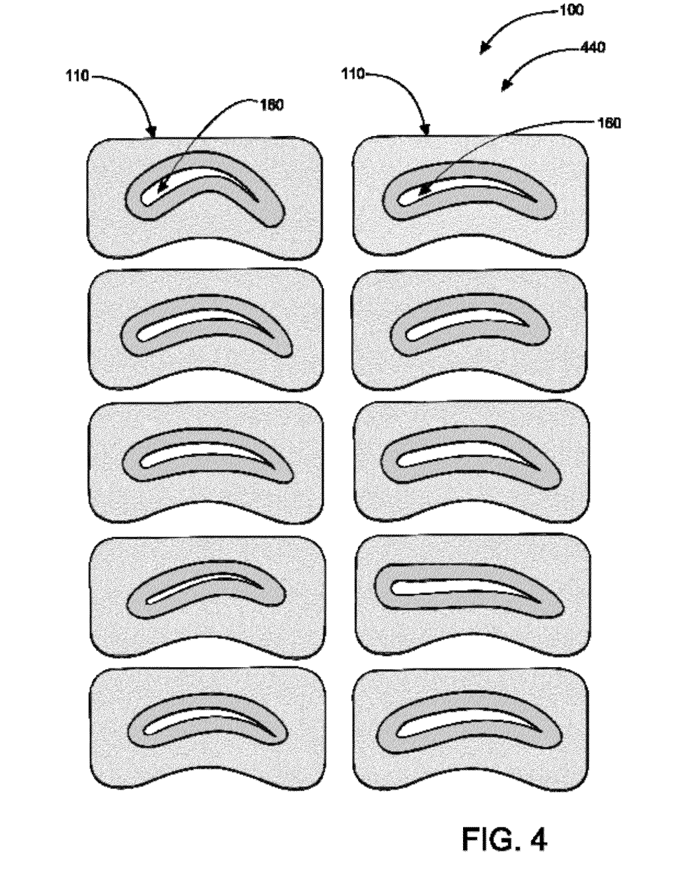

Referring now to FIG. 4 showing a perspective view illustrating kit 440 of self eyebrow waxing and shaping system 100 according to an embodiment of the present invention.

Self eyebrow waxing and shaping system 100 may be sold as kit 440 comprising: a plurality of left eyebrow shaping stencil template strip(s) 120 having various pre-shaped eyebrow cutout designs 160; a plurality of right eyebrow shaping stencil template strip(s) 130 having various pre-shaped eyebrow cutout designs ; and a set of user instructions. Other pre-shaped eyebrow cutout designs 160 may be included within kit 440 of varying sizes or the like for use with different versions. Self eyebrow waxing and shaping system 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized colors/designs, components may be sold separately, etc., may be sufficient.

Pre-shaped eyebrow cutout 160 is located in central portion 118 on the eyebrow shaping stencil template strip(s) 110. Pre-shaped eyebrow cutout 160 includes left pre-shaped eyebrow cutout 162 and right pre-shaped eyebrow cutout 164, as previously mentioned. Each of left pre-shaped eyebrow cutout 162 and right pre-shaped eyebrow cutout 164 comprise a different shape and design on each eyebrow shaping stencil template strip(s) 110. They are designed to be symmetrical from side to side to give a desired final appearance.

Left pre-shaped eyebrow cutout 162 and right pre-shaped eyebrow cutout 164 preferably comprises ten different eyebrow shape variations on eyebrow shaping stencil template strip(s) 110. Kit 440 may include more or less than ten different eyebrow shape variations on eyebrow shaping stencil template strip(s) 110. Further, kit 440 may include more than one of each eyebrow shape variation (pre-shaped eyebrow cutout 160). Pre-shaped eyebrow cutout 160 is preferably arched and contoured to a natural shape of eyebrow 170 with an outer end of eyebrow 170 being tapered.

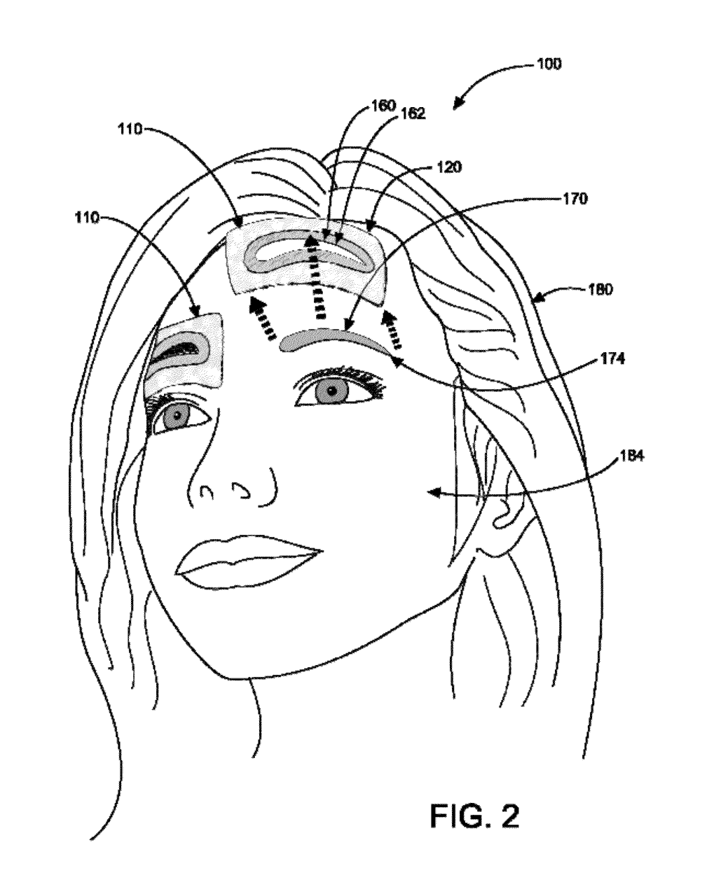

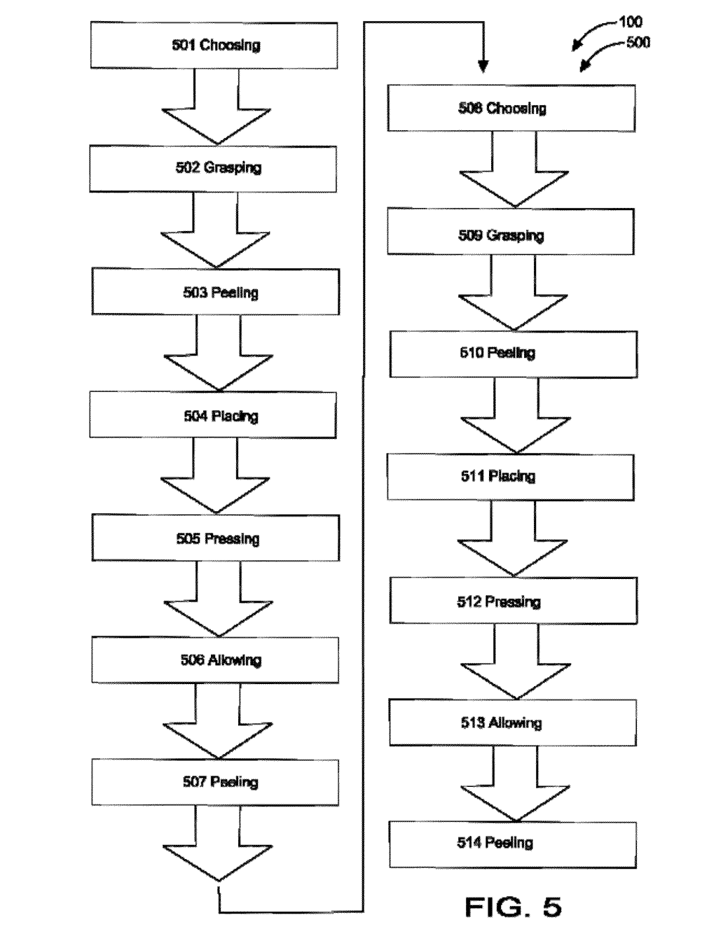

Referring now to FIG. 5, a flowchart 550 illustrating a method of using (method of use 500) self eyebrow waxing and shaping system 100 according to an embodiment of the present invention of FIGS. 1-4.

A method of using (enabling method of use 500) self eyebrow waxing and shaping system 100 preferably comprises the steps of: step one 501 choosing left eyebrow shaping stencil template strip 120 with a desired left pre-shaped eyebrow cutout design 162; step two 502 grasping elongated corner 114 of eyebrow shaping stencil template strip(s) 110; step three 503 peeling away protective peel off sheet 150; step four 504 placing eyebrow shaping stencil template strip(s) 110 over left eyebrow 174; step five 505 pressing eyebrow shaping stencil template strip(s) 110 onto skin around left eyebrow 174 of user 180 where unwanted eyebrow hair is located; step six 506 allowing adhesive wax backing 140 to grab the unwanted eyebrow hair; step seven 507 quickly peeling eyebrow shaping stencil template strip(s) 110 away from eyebrow 170 to reveal a newly shaped eyebrow; step eight 508 choosing right eyebrow shaping stencil template strip(s) 130 with a desired right pre-shaped eyebrow cutout design 164; step nine 509 grasping elongated corner 114 of eyebrow shaping stencil template strip(s) 110; step ten 510 peeling away protective peel off sheet 150; step eleven 511 placing eyebrow shaping stencil template strip(s) 110 over right eyebrow 178; step twelve 512 pressing eyebrow shaping stencil template strip(s) 110 onto skin around right eyebrow 178 of user 180 where unwanted eyebrow hair is located; step thirteen 513 allowing adhesive wax backing 140 to grab unwanted eyebrow hair; and step fourteen 514 quickly peeling eyebrow shaping stencil template strip(s) 110 away from eyebrow 170 to reveal a newly shaped eyebrow. In this way the system may be used from side to side; however the order may be changed such that it doesn't require steps to be done in a side to side order of events.

It should be noted that steps eight 508 through step fourteen 514 are optional steps and may not be implemented in all cases. Optional steps of method 500 are illustrated using dotted lines in FIG. 5 so as to distinguish them from the other steps of method 500.

It should be noted that the steps described in the method of use can be carried out in many different orders according to user preference. The use of “step of” should not be interpreted as “step for”, in the claims herein and is not intended to invoke the provisions of 35 U.S.C. §112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A self eyebrow waxing and shaping system comprising:
eyebrow shaping stencil template strips having;
a plurality of left eyebrow shaping stencil template strips;
a plurality of right eyebrow shaping stencil template strips;
an adhesive wax backing;
a protective peel off sheet; and
a pre-shaped eyebrow cutout;
wherein each of said left eyebrow shaping stencil template strips comprise said adhesive wax backing, said protective peel off sheet, and said pre-shaped eyebrow cutout;
wherein each of said right eyebrow shaping stencil template strips comprise said adhesive wax backing, said protective peel off sheet, and pre-shaped eyebrow cutout;
wherein said pre-shaped eyebrow cutout is located in a central portion on said eyebrow shaping stencil template strips;
wherein each of said pre-shaped eyebrow cutouts comprise a different shape and design on each eyebrow shaping stencil template strip;
wherein said adhesive wax backing is located on a bottom side of said eyebrow shaping stencil template strip;
wherein said adhesive wax backing on said eyebrow shaping stencil template strip removes unwanted hair and creates a desired shape of said eyebrow as per shape of said pre-shaped eyebrow cutout;
wherein said protective peel off sheet protects said adhesive wax backing disposed upon said bottom side of said eyebrow shaping stencil template strip and is peeled off prior to a user using said eyebrow shaping stencil templates; and
wherein said self eyebrow waxing and shaping system is usable to independently create and maintain symmetrical eyebrow shapes.

2. The self eyebrow waxing and shaping system of claim 1 wherein said eyebrow shaping stencil template strip comprises an elongated corner to permit ease of removing said protective peel off sheet thereby exposing said adhesive wax backing.

3. The self eyebrow waxing and shaping system of claim 1 wherein said eyebrow shaping stencil template strips are transparent beige thereby permitting said user to visualize how much of said eyebrow will be removed upon completion of said self eyebrow waxing.

4. The self eyebrow waxing and shaping system of claim 1 wherein said eyebrow shaping stencil template strips are rectangular in shape.

5. The self eyebrow waxing and shaping system of claim 1 wherein left said eyebrow shaping stencil template strip is removeably-placed over a left said eyebrow of said user to alter a shape of left said eyebrow.

6. The self eyebrow waxing and shaping system of claim 1 wherein right said eyebrow shaping stencil template strip is removeably-placed over a right said eyebrow of said user to alter a shape of right said eyebrow.

7. The self eyebrow waxing and shaping system of claim 1 wherein said pre-shaped eyebrow cutout is arched and contoured to a natural shape of said eyebrow with an outer end of said eyebrow being tapered.

8. The self eyebrow waxing and shaping system of claim 1 wherein said eyebrow shaping stencil template strips comprise a sheet of waxing material with said adhesive backing.

9. The self eyebrow waxing and shaping system of claim 1 wherein said pre-shaped eyebrow cutout comprises ten different eyebrow shape variations on eyebrow shaping stencil template strip.

10. A self eyebrow waxing and shaping system comprising:
rectangular eyebrow shaping stencil template strips having;
a plurality of left eyebrow shaping stencil template strips;
a plurality of right eyebrow shaping stencil template strips;
an adhesive wax backing;
a protective peel off sheet; and
a pre-shaped eyebrow cutout;
wherein said eyebrow shaping stencil template strips comprise a sheet of waxing material with said adhesive backing;
wherein each of left said eyebrow shaping stencil template strips comprise said adhesive wax backing, said protective peel off sheet, and said pre-shaped eyebrow cutout;
wherein each of right said eyebrow shaping stencil template strips comprise said adhesive wax backing, said protective peel off sheet, and said pre-shaped eyebrow cutout;
wherein left said eyebrow shaping stencil template strip is removeably-placed over a left said eyebrow of said user to alter a shape of left said eyebrow;
wherein right said eyebrow shaping stencil template strip is removeably-placed over a right said eyebrow of said user to alter a shape of right said eyebrow;
wherein said eyebrow shaping stencil template strip comprises an elongated corner to permit ease of removing said protective peel off sheet thereby exposing said adhesive wax backing;
wherein said eyebrow shaping stencil template strips are transparent beige thereby permitting said user to visualize how much of said eyebrow will be removed upon completion of said self eyebrow waxing;
wherein said pre-shaped eyebrow cutout is located in a central portion on said eyebrow shaping stencil template strips;
wherein each of said pre-shaped eyebrow cutouts comprise a different shape and design on each said eyebrow shaping stencil template strip;
wherein said pre-shaped eyebrow cutout comprises ten different eyebrow shape variations on said eyebrow shaping stencil template strip;
wherein said pre-shaped eyebrow cutout is arched and contoured to a natural shape of said eyebrow with an outer end of said eyebrow being tapered;
wherein said adhesive wax backing is located on a bottom side of said eyebrow shaping stencil template strip;
wherein said adhesive wax backing on said eyebrow shaping stencil template strip removes unwanted hair and creates a desired shape of said eyebrow as per shape of said pre-shaped eyebrow cutout;
wherein said protective peel off sheet protects said adhesive wax backing disposed upon said bottom side of said eyebrow shaping stencil template strip and is peeled off prior to said user using said eyebrow shaping stencil templates; and
wherein said self eyebrow waxing and shaping system is usable to independently create and maintain symmetrical eyebrow shapes.

11. The self eyebrow waxing and shaping system of claim 10 further comprising a kit, wherein said kit comprises: a plurality of left said eyebrow shaping stencil template strips in various pre-shaped eyebrow cutout designs; a plurality of right said eyebrow shaping stencil template strips in various pre-shaped eyebrow cutout designs; and a set of user instructions.

12. A method of using a self eyebrow waxing system comprising the steps of:
choosing a left eyebrow shaping stencil template strip with a desired left pre-shaped eyebrow cutout design;
grasping an elongated corner of eyebrow shaping stencil template strip;
peeling away a protective peel off sheet;
placing eyebrow shaping stencil template strip over a left said eyebrow;
pressing said eyebrow shaping stencil template strip onto skin around said left said eyebrow of a user where unwanted eyebrow hair is located;
allowing adhesive wax backing to grab said unwanted eyebrow hair; and
quickly peeling said eyebrow shaping stencil template strip away from said eyebrow to reveal a newly shaped eyebrow.

13. The method of using the self eyebrow waxing and shaping system of claim 12 further comprising the steps of:
choosing a right eyebrow said shaping stencil template strip with a desired right pre-shaped eyebrow cutout design;
grasping an elongated corner of eyebrow shaping stencil template strip;
peeling away said protective peel off sheet;
placing said eyebrow shaping stencil template strip over a right said eyebrow;
pressing said eyebrow shaping stencil template strip onto skin around said right said eyebrow of said user where said unwanted eyebrow hair is located;
allowing adhesive wax backing to grab said unwanted eyebrow hair; and
quickly peeling said eyebrow shaping stencil template strip away from said eyebrow to reveal a newly shaped eyebrow.
